(19) United States
(12) Patent Application Publication
(10) Pub. No.: US 2003/0036562 A1
(43) Pub. Date: Feb. 20, 2003

(54) Water-soluble dendrimeric fullerene as anti-HIV therapeutic

(76) Inventors: Raymond F. Schinazi, Decatur, GA (US); Michael Brettreich, Los Angeles, CA (US); Andreas Hirsch, Rathsberg (DE)

Correspondence Address: Raymund F Eich, Williams Morgan & Amerson P C, 7676 Hillmont Suite 250, Houston, TX, 77040, US

(21) Appl. No.: 09/853,202

(22) Filed: May 11, 2001

(51) Int. Cl. A61K 31/192; A61K 31/235

(52) U.S. Cl. 514/569; 514/533

(57) ABSTRACT

A composition comprising:

(a) a pharmaceutically effective amount of water-soluble fullerene compounds of one or more of the formulae I-VI:

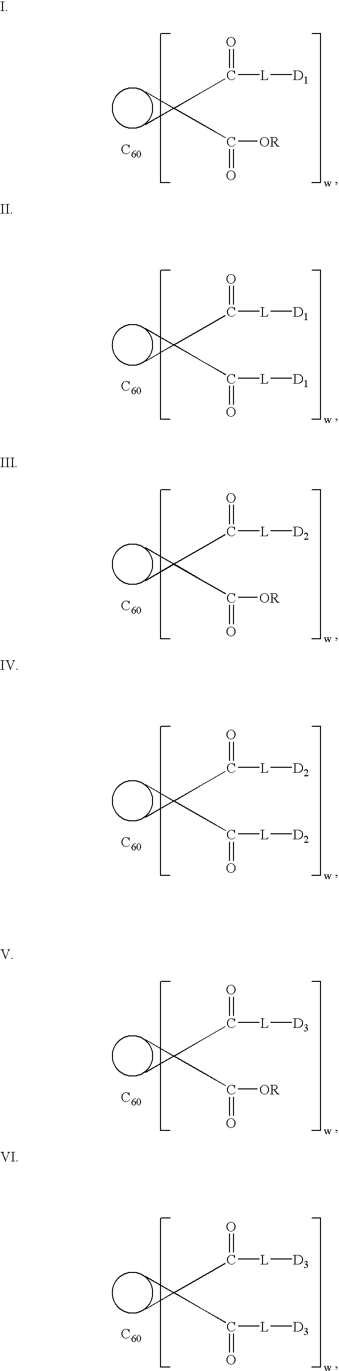

where L is a linker of the formula

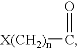

X is NH or O,

R is H or lower alkyl having 1-4 carbon atoms,

n is 1-6, and

D₁ is a dendron of the formula

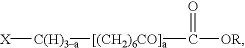

D₂ is a dendron of the formula

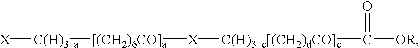

D₃ is a dendron of the formula

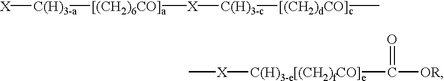

a, c and e are the same or different and each is 1 or 2, b, d and f are the same or different and each is 1-6 and w is 1-6, mixtures thereof, and water soluble salts thereof, and

(b) a pharmaceutically acceptable carrier.

The composition is used to treat HIV.

### FIELD OF THE INVENTION

The present invention relates to compositions comprising pharmaceutically effective amounts of water-soluble fullerene derivatives for treatment of HIV and to methods for using such compositions in the treatment of HIV.

### BACKGROUND OF THE INVENTION

Since the discovery of fullerene-C₆₀ in 1985 by Kroto et al. (1) the remarkable properties of fullerenes have been extensively studied and documented (2). Naturally occurring fullerenes have been found in burned carbon sources such as Chinese calligraphy ink (3) and in the KT-boundary of the earth (4). Recently, fullerenes also have been reported to occur in extraterrestrial objects such as carbonaceous meteors (5).

Over the last few years, several medical and biological applications for fullerene derivatives have been explored, with encouraging results (6, 7). Many of these studies employed four “classic” water-soluble derivatives (8, 9, 10). More recently, other types of water-soluble compounds have also been synthesized and characterized (11). The expanding potential for the use of fullerene derivatives in biological situations indicates a need for antibodies that could be used to detect them in blood or tissue. Monoclonal antibodies have been produced for numerous antigens/molecules of interest, (12) although most have been directed towards “natural” biological and organic substances. The remarkable structure of buckminsterfullerenes raised the question of whether the immune system could provide a response to such materials. In fact, anti-fullerene antibodies can be prepared using very standard techniques such as conjugation of the small molecule hapten (the fullerene) to a protein (13, 14).

Computational study of this antibody, using molecular modeling and coordinates from the X-ray crystal structure (FIG. 3A) (14) shows the location of the recognition site for the fullerene-termed the CDR region or complementarity-determining region. Another aspect involves the binding selectivity for four different fullerene drug molecules. Water soluble fullerene compound shown in FIG. 1B binds to the anti-C₆₀ antibody.

Of particular relevance to the present work was the discovery by Friedman et al. (15) that the water-soluble methanofullerene derivative of FIG. 1A, compound “1”, was a competitive inhibitor of recombinant protease specific for human immunodeficiency virus (HIV-P) with a Kᵢ of 5.3 μM. Friedman et al. anticipated that the C₆₀ sphere should fit into the hydrophobic cavity of HIV-P. Using computer modeling, they were able to fit a minimized structure of C₆₀ into the enzyme active site. Compound 1 was evaluated by Schinazi et al. (16, 17) for antiviral activity in acutely HIV-1 and HIV-2 infected human peripheral blood mononuclear cells (PBMC) and found to have a median effective concentration (EC₅₀) of 7.3 μM, and 5.5 μM, respectively. Compound 1 was also active in chronically infected H9 cells (EC₅₀=10.8 μM); selective activity in these cells is considered a hallmark of all protease inhibitors. Schinazi et al. also reported that the compound had anti-HIV-P activity at a concentration comparable to the antiviral activity observed in infected lymphocytes. It was also shown that compound 1 has direct virucidal activity (18), and similar antiviral activity against AZT-susceptible, as well as AZT-resistant HIV-1 in infected PBMC. No cytotoxicity was observed up to 100 μM in uninfected slowly dividing PBMC or rapidly dividing H9, Vero, or CEM (human lymphoblastoid) cells, under conditions where AZT is cytotoxic in all but the first cell line. Furthermore, when compound 1 was administered intraperitoneally to mice at doses up to 50 mg/kg per day for 6 days, all animals steadily gained weight and none died up to two months after initial treatment.

Subsequently, a large number of fullerene derivatives were shown to have anti-HIV-1 activity in the low micromolar range with no measurable toxicity (IC₅₀>100 μM) in human PBMC and Vero cells from African Green monkeys (19). Based upon a theoretical model involving hydrophobic desolvation, two designed fullerene derivatives were shown to bind somewhat more tightly to HIV-P (20).

The in vivo behavior of C₆₀ derivatives has also been studied (22). Two conclusions were drawn from the investigation: 1) the two studied C₆₀ derivatives are only toxic at high doses, in contrast with unmodified C₆₀ which is non toxic even at high doses (23), 2) the water soluble C₆₀ derivatives can be efficiently absorbed after oral administration and readily eliminated through the kidneys, in contrast with unmodified C₆₀. Of particular import is that the compound of FIG. 1B (compound 2) has an LD₅₀˜700 mg/kg and is 30% orally absorbed.

PCT International Application No. WO 99/43358, published Sep. 2, 1999, discloses dendrimeric fullerene derivatives in which the fullerene is linked to at least one dendron. Each dendron has at least one protic group which confers water solubility on the derivatives. Dendrimers consist of two or more highly ordered, three-dimensional dendritic arrays called “dendrons”. Dendrons may be designed by selection of a “molecular seed” (core) from which the dendritic branching arrays are grown (24).

U.S. Pat. No. 5,688,486 discloses fullerenes that can be used as carriers for diagnostic or therapeutic agents, especially diagnostic contrast agents. U.S. Pat. No. 5,811,460 discloses water-soluble fullerenes known to have anti-viral properties. U.S. Pat. No. 6,204,391 B1 discloses a water-soluble derivative of buckministerfullerene (C₆₀) having antiviral and virucidal properties used to inhibit human retroviral replication and infections.

### OBJECTS OF THE INVENTION

It is a primary object of the invention to provide a water-soluble fullerene derivative which is therapeutically effective against strains of the HIV that are resistant to other compounds currently used as components of HIV drug “cocktails”.

It is another and related object of the invention to provide a water-soluble fullerene derivative which is therapeutically effective against strains of the HIV and which may be used in conjunction with other compounds as part of a drug “cocktail”.

### SUMMARY OF THE INVENTION

The invention is in compositions comprising:

(a) a pharmaceutically effective amount of water-soluble fullerene compounds of one or more of the formulae I-VI:

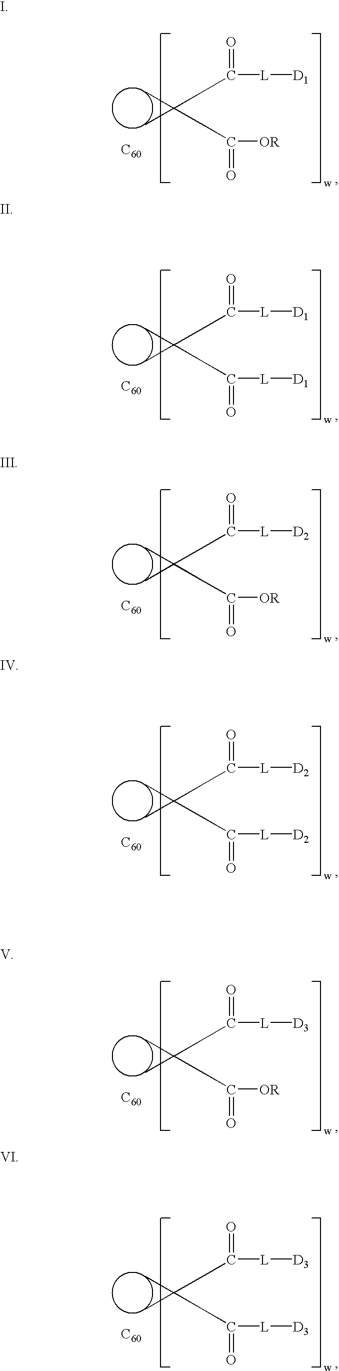

where L is a linker of the formula

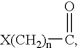

X is NH or 0,

R is H or lower alkyl having 1-4 carbon atoms,

n is 1-6, and

D₁ is a dendron of the formula

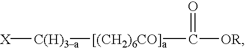

D₂ is a dendron of the formula

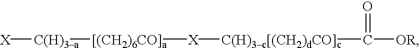

D₃ is a dendron of the formula

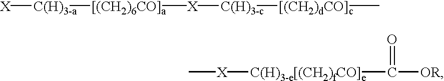

a, c and e are the same or different and each is 1 or 2, b, d and f are the same or different and each is 1-6 and w is 1-6, or a mixture thereof, or water soluble salts thereof, and

(b) a pharmaceutically acceptable carrier.

The compositions of the invention are useful in the treatment of viral diseases, including HIV.

### BRIEF DESCRIPTION OF THE FIGURES

FIG. 1A shows a water-soluble methanofullerene derivative (compound 1) (15).

FIG. 1B shows a water-soluble dendrimeric derivative of C₆₀ (“generation 2”, or D₂, dendrofullerene, compound 2) (9).

FIG. 2 shows binding of water-soluble fullerene compounds 2 (K_{d}=1.2 mg/ml) (FIG. 1B), A (C3-carboxyfullerene, K_{d}=0.25 mg/ml), B (D3-carboxyfullerene, K_{d}=0.038 mg/ml), and C (fullerenol, K_{d}=0.75 mg/ml) to Anti-C₆₀ (measured inhibition of bovine thyroglobulin-fullerene conjugate binding by test compound) (14).

FIG. 3A shows C₆₀ in the binding site of anti-C₆₀ antibody (14).

FIG. 3B shows the dendrimer of FIG. 1B docked in HIV protease.

FIG. 4A shows “generation 1” (D₁) dendron.

FIG. 4B shows “generation 2” (D₂) dendron.

FIG. 4C shows “generation 3” (D₃) dendron.

FIG. 5 shows the steps in the construction of a dendron.

FIG. 6 shows the amplification stages of a dendrimer.

### DETAILED DESCRIPTION OF THE INVENTION

The invention is in compositions comprising: (a) a pharmaceutically effective amount of water-soluble fullerene compounds of one or more of the formulae I-VI.

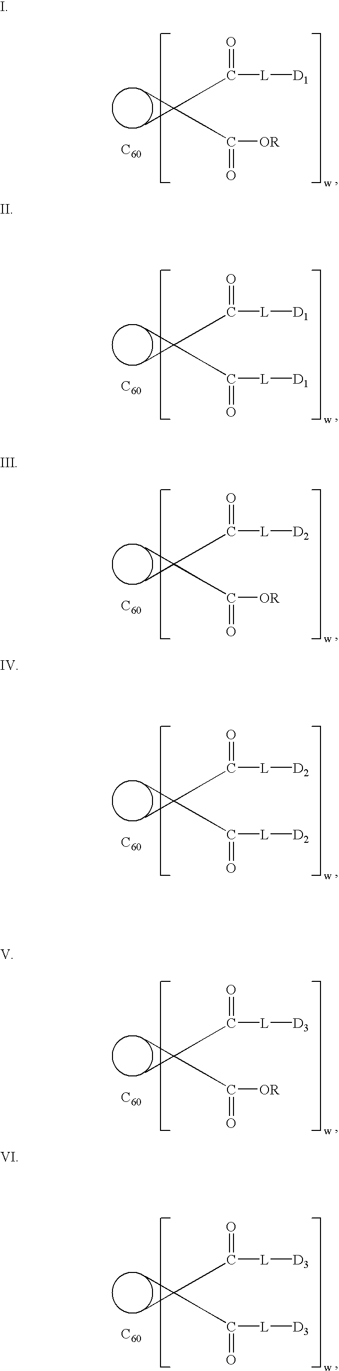

where L is a linker of the formula

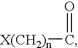

X is NH or O,

R is H or lower alkyl having 1-4 carbon atoms,

n is 1-6, and

D₁ is a dendron of the formula

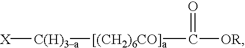

D₂ is a dendron of the formula

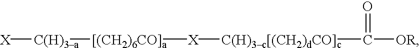

D₃ is a dendron of the formula

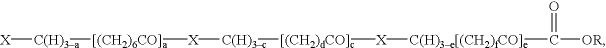

a, c and e are the same or different and each is 1 or 2, b, d and f are the same or different and each is 1-6 and w is 1-6, or mixtures thereof, or water soluble salts thereof, and

(b) a pharmaceutically acceptable carrier.

The pharmaceutically acceptable carrier may be any carrier conventionally used in the art, including liposomes.

In one embodiment of the invention, the compositions have virucidal properties against a human retrovirus. In another embodiment of the invention, the compositions are useful in the treatment of HIV. In yet another embodiment of the invention, the compositions are used to treat strains of HIV such as M184V, HIV-1/LAI, xxBRU, M184V/T215Y, and 215/41. In other embodiments, the compositions are used to treat strains of HIV which are 3TC resistant, AZT/3TC resistant or AZT resistant.

In an embodiment of the invention, compositions are administered to patients infected with the virus in dosages of 5-25 mg/day. In another embodiment of the invention, the dosage of 5-25 mg/day is administered orally. In a preferred embodiment of the invention, the compositions contain the second generation dendrimeric fullerene derivative.

In an embodiment of the invention, the structure of the first generation dendrimeric fullerene derivative (D₁) is:

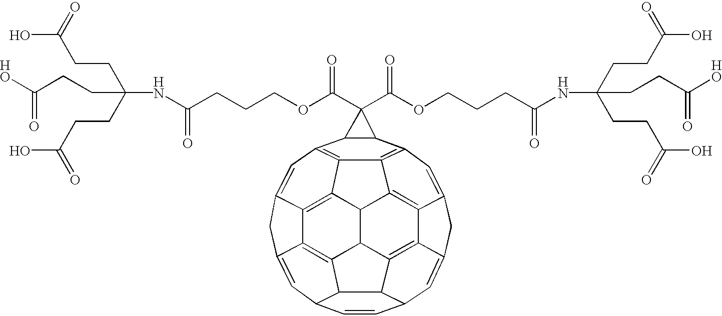

In another embodiment of the invention, the structure of the second generation dendrimeric fullerene derivative (D₂) is:

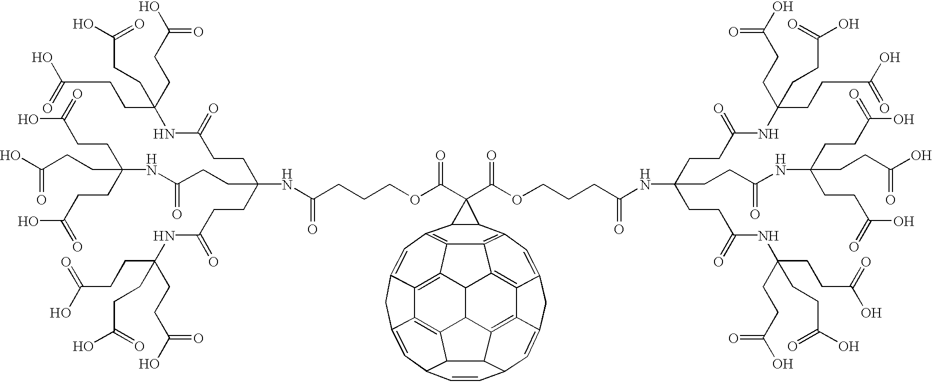

In another embodiment of the invention, the structure of the third generation dendrimeric fullerene derivative (D₃) is:

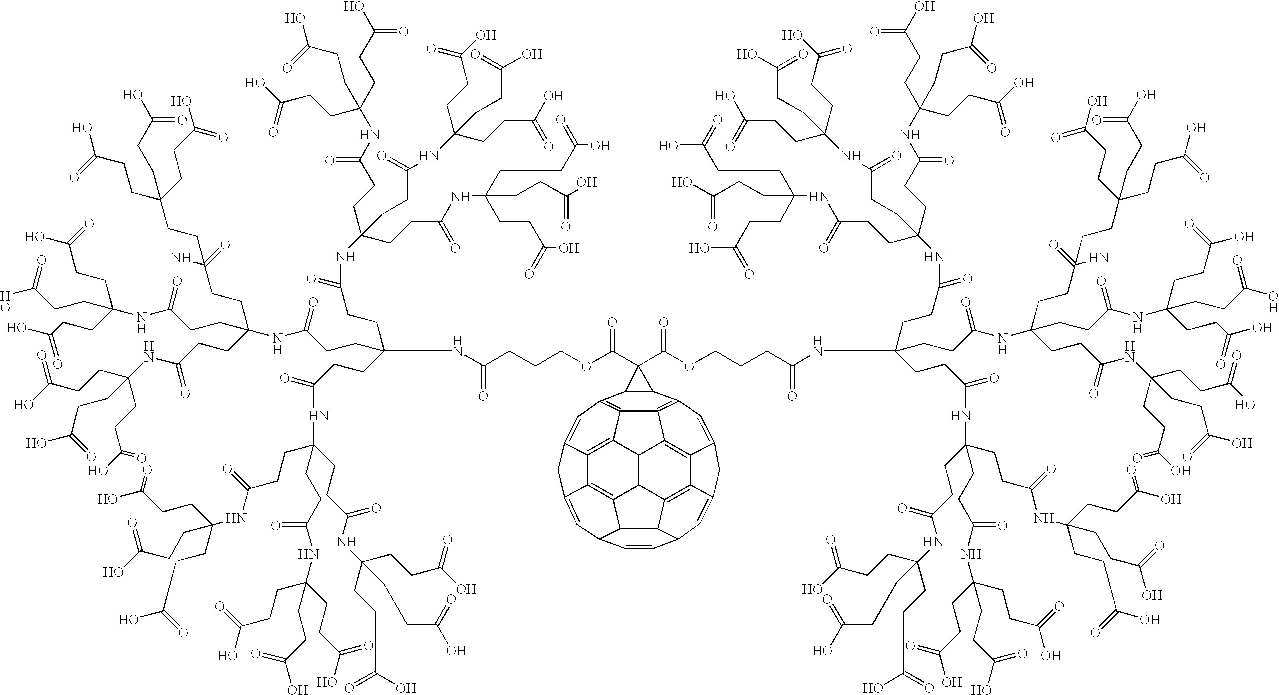

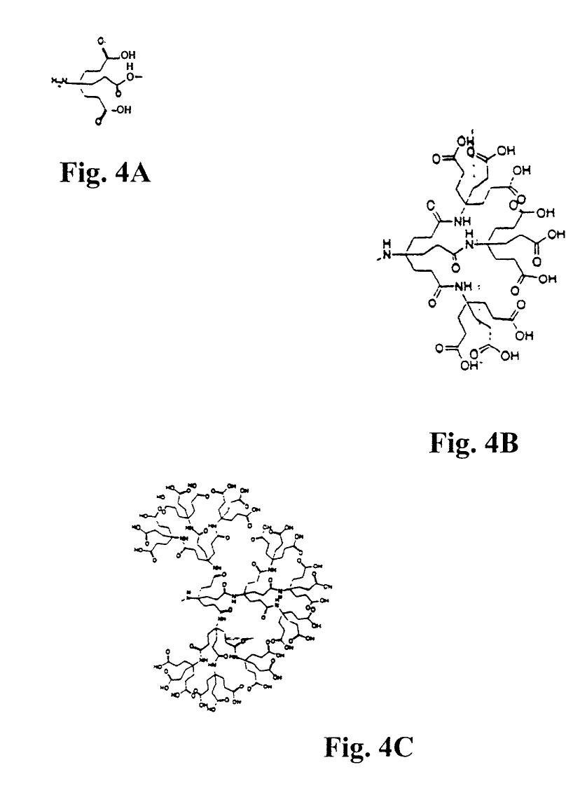

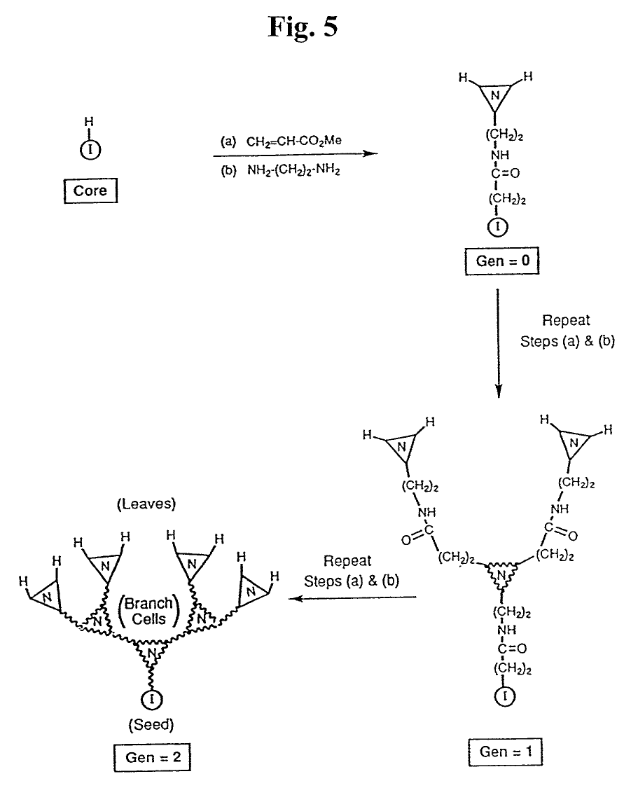

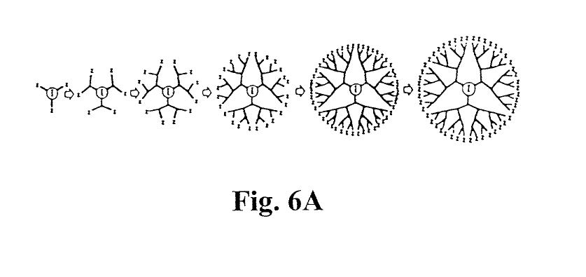

Dendrimers are prepared by a sequential, repetitive technique (25). Each complete reaction sequence results in a new “generation” with a larger diameter, three times the number of reactive sites, and approximately three times the molecular weight of the preceeding generation. FIG. 4 shows a “generation 1” dendron (FIG. 4a), “generation 2” dendron (FIG. 4b) and “generation 3” dendron (FIG. 4c). FIG. 5 illustrates construction of a dendrimer by showing the growth of a mon-, di-, and tri-dendron from simple molecular seeds. FIG. 6 shows amplification stages defined by the concentric layers of branch junctures that describe the growth stages (generations) and interior of the dendrimer.

### EXAMPLES

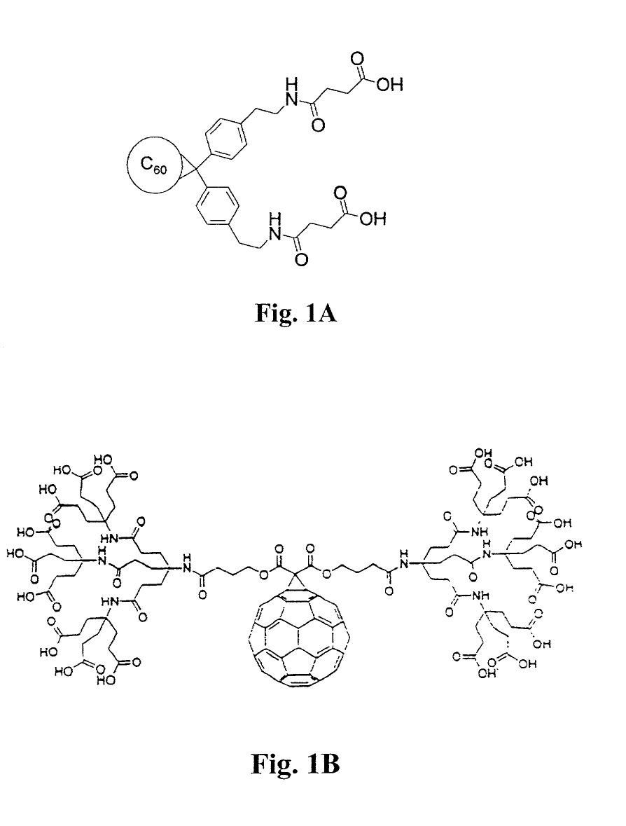

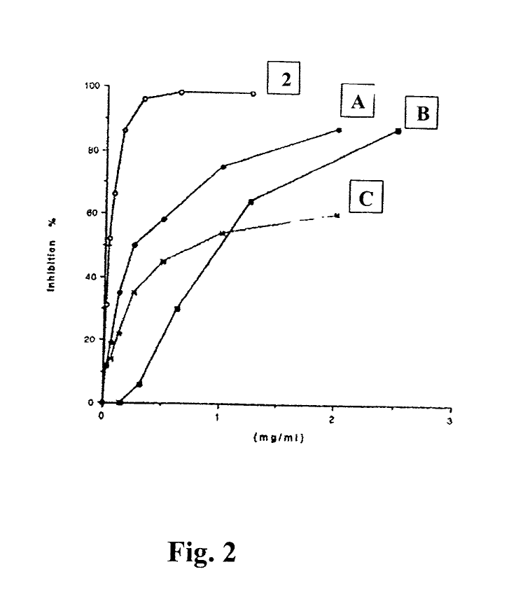

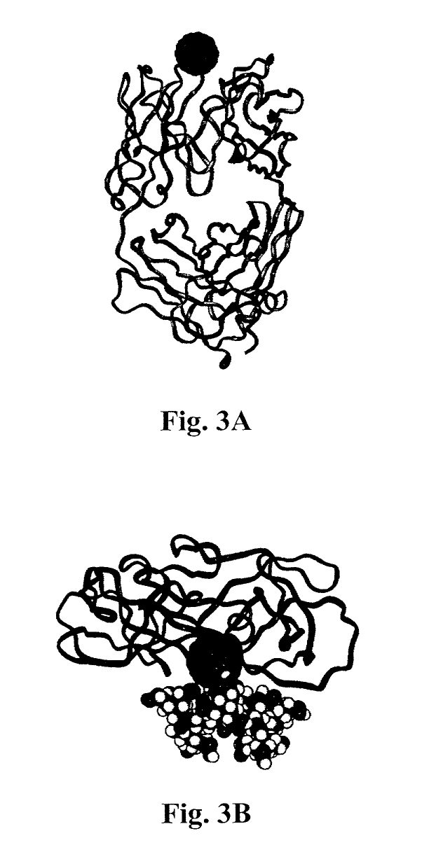

Example 1 (below) shows that a highly water-soluble dendrimeric derivative of C₆₀ (FIG. 1B, compound 2) with 18 carboxylic acid groups was found to be active in primary human lymphocytes acutely infected with HIV-1_{LAI} with an EC₅₀ of 0.22 μM, and showed no toxicity up to 100 μM in human PBM, Vero and CEM cells. The Kᵢ for inhibition of cloned HIV-protease was 0.10±0.01 μM with an E/I stoichiometry of 1:12. The activity of the fullerene dendrimer against HIV reverse transcriptase was 0.9 μM. The fullerene dendrimer was also active against mutant molecular infectious clones of HIV-1 which are resistant to AZT and/or 3TC, drugs that are widely used in AIDS therapy. Computer modeling indicates that the hydrophilic side arms of the dendrimer protrude from the hydrophobic cavity of HIV-protease (FIG. 3B), where the fullerene sphere blocks access to the enzyme active site. This dendrimer is one of the most active anti-HIV fullerene derivatives yet discovered.

The formula of the dendrimeric derivative (compound 2, FIG. 1B) is C₁₅₁H₁₃₄N₈O₄₈. The solubility in water of the dendrimeric derivative, at pH 7.4, is 34 mg per ml (8.7 mg per ml C₆₀).

### EXAMPLE 1

### Evaluation of the Anti-HIV Potency of a Water-soluble Dendrimeric Fullerene

The invention is in the discovery of one of the most active antiviral fullerene derivatives studied to date, namely a highly water-soluble (34 mg/ml at pH 7.4) dendrimeric derivative of C₆₀, FIG. 1B (compound 2) with 18 carboxylic acid groups (21). An aqueous solution of compound 2 was tested in primary human lymphocytes acutely infected with HIV-1_{LAI}, where it had an EC₅₀, of 0.22 μM. The EC₅₀ against several molecular infectious clones of HIV-1 which are resistant to the well-known viral inhibitors 3TC and/or AZT were as follows: xxBRU, 0.19 μM; M184V (methionine changed to valine at residue 184 in the reverse transcriptase 0.052 μM; T215Y/M41L, 0.97 μM; and M184V/T215Y, 0.58 μM. Thus, all these mutant viruses are susceptible to this compound at low concentrations. The dendrimer had no apparent cytotoxicity in human PBM, Vero or CEM cells, up to 100 μM.

The IC₅₀ value of compound 2 against recombinant HIV-1 p66/51 reverse transcriptase in the absence of exogenous protein was found to be 0.9 μM. The Kᵢ for inhibition of HIV-P, analyzed using a chromogenic substrate of compound 2, was found to be 0.1 μM with an E/I stoichiometry of 1:12. Furthermore, the potency of compound 2 in human PBM cells infected with HIV is similar to the activity observed with the HIV-P.

Because of the size and structure of the compound of FIG. 1A (compound 1), it is likely that the hydrophilic side arms protrude from the hydrophobic cavity of HIV-P into the surrounding medium. Thus, it is unlikely that the activity involves direct interaction with the active site aspartyl residues within the cavity of the enzyme, but rather that the fullerene moiety blocks access to the enzyme active site. Computer modeling studies of compound 2 docked in HIV-P (see FIG. 3B) using Insight II support this conclusion.

The model for HIV-P was obtained from an X-ray crystal structure (RSCB Protein Data Bank identification 1AID). It should be noted that the structure of HIV-P is a homodimer, which is C₂-symmetric about the theoretical binding site. The fullerene molecule was docked at the binding pocket and minimization was performed until the calculation converged at RMS=0.001. In the process of docking compound 2 into HIV-P, it was noticed that the ball-shaped structure fits very well into the pocket, while the dendrimer side chains protrude from the binding pocket outward, loosely associating themselves with the outer surface of the protein's binding site region (FIG. 3B).

The calculated intermolecular energy for the minimized model was negative, indicating the presence of favorable interaction between compound 2 and the protein. Both holding the HIV-P structure fixed in space and allowing it to relax its structure resulted in a minimized model that shows tight binding of the fullerene dendrimer. The relaxed form has the atoms of the binding site noticeably curved around the fullerene molecule, indicating strong binding affinity.

Preliminary pharmacological studies demonstrated that compound 2 may be orally absorbed and is excreted in the urine (22).

### Example 2

### Antiviral Activity and Cytotoxicity of DSW Compounds

Anti-HIV activity and cytotoxicity of the dendrimeric fullerene derivative compound 2, (FIG. 1B) was tested. The compound showed significant anti-HIV activity and no cytotoxicity in PBM or Vero cells, but it is slightly toxic in CEM cells.

Compound 2 was warmed at 37° C. to solubilize it, but some material remained in suspension.

TABLE 1
Antiviral Activity and Cytotoxicity of Compounds

| | EC50, | EC90, | PBM (MTT) | Vero (MTT) | CEM (MTT) |
|---|---|---|---|---|---|
| Code | μM | μM | IC50, μM | IC50, μM | IC50, μM |
| Sample A | 0.021 | 0.13 | >100 (−55) | >10 (−24) | 54.5 |
| Sample B | 0.15 | 1.19 | >100 (−14) | >100 (−19) | 47.6 |
| Sample C | 0.096 | 1.23 | >100 | >100 | ND |
| Average* | 0.12 | 1.21 | >100 | >100 | 51.1 |

Average* denotes the average of DSW-057a testing in T25 flasks only. Data in parentheses indicates the % inhibition at 100 μM. EC is “effective concentration”. EC₅₀ is “median effective concentration”.

Studies with compound 2 (FIG. 1B) and several strains of HIV, including the 3TC-resistant strain (M184V) are shown in Table II. Table III shows cytotoxicity in different cells. The activity of compound 2 is 52 nM against M184, whereas 3TC is inactive (>100) against M184V. Compound 2 is also very active against the other resistant strains.

TABLE II(A)
Effect of Compound 2 on Viruses in Human PBM Cells
Antiviral activity of fullerene derivative Compound 2 in human peripheral
blood mononuclear cells

| | | | Fold increase: | |
|---|---|---|---|---|
| Virus | EC50 μM | EC90 μM | F1 50 | F1 90 |
| HIV-1/LAI | 0.22 | 1.80 | — | — |
| xxBRU* | 0.19 | 1.1 | — | — |
| M184V* (3TC resistant) | 0.052 | 0.7 | 0.3 | 0.6 |
| M184V/T215Y* | | | | |
| (AZT/3TC res.) | 0.58 | 2 | 3 | 2 |
| 215/41 | 0.97 | 2.75 | 5 | 3 |
| (AZT resistant) | | | | |

A five fold or less increase in EC50 or EC90 is not considered significant.
*Molecular infectious clone
Averages for data are italicized in the tables.
FI(Fold increase) EC50 = EC50 data from resistant virus/EC50 data from HIV-1/LA1 or xxBRU
FI(Fold increase) EC90 = EC90 data from resistant virus/EC90 data from HIV-1/LAI or xxBRU

TABLE 11(B)
Cytotoxicity of Compound 2 in Different Cells
CYTOTOXICITY

| | PBM (MTT) | Vero (MTT) | CEM (MTT) |
|---|---|---|---|
| | IC50, μM | IC50, μM | IC50, μM |
| Compound 2 | >100 | >100 | 51.1 |

MTT is an colorimetric cytotoxicity assay. The assay measures mitochondrial function in cells.

The unique action of compound 2 is also revealed in reverse transcriptase (RT) activity (Table III). The IC₅₀ for AZT is 0.021 against RT, vs. the DSW-057 IC₅₀ of 0.89 using a poly (rA)ₙ oligo (dT)₁₂ template primer. Therefore, compound 2 may have a dual mechanism for anti-HIV activity. Studies were also performed using HIV-1 protease. The Kᵢ value for compound 2 was found to be 0.1 μM and an E/I stoichiometry of 1:12 using HIV-1 protease.

TABLE III
Inhibition of p66/51 RT by AZTTP & Compound 2

Enzyme: p66/51 1 unit/rxn, lot 3808004 (02).
Reaction mix: 100 μM: 100 mM Tris-HCL, pH 8.0, 50 mM KCL, 2 mM MgCl2, 0.05 U/ml (rA)n.(dT)12-18, 5 mM
DTT, 9H dTTP (1 μM, 65 Ci/mmol, lot 227-186-060).
*Harvest/count with Packard 9600 Harvester/Direct Bela Counter.

| Control | cpm | average | -bkgrd | Background | cpm | average |
|---|---|---|---|---|---|---|
| | 16,825 | 16,548 | 16,422 | | 56 | 126 |
| | 16,305 | | | | 49 | |
| | 16,513 | | | | 273 | |

| | Concentration | | | | | | | | |
|---|---|---|---|---|---|---|---|---|---|
| DRUG | (μM) | cpm | average | -bkgrd | % Inhibition | 1C 50, μM | 1C 90, μM | R | m |
| AZTTP | 10 | 261 | 212 | 86 | 99.6 | 0.021 | 0.27 | 0.998 | 0.848 × 0.33 |
| SB 3/98 | | 163 | | | | | | | |
| 608 μM | 1 | 529 | 611 | 485 | 97.0 | | | | |
| | | 692 | | | | | | | |
| | 0.1 | 4,517 | 4,612 | 4,486 | 72.7 | | | | |
| | | 4,707 | | | | | | | |
| | 0.01 | 11,868 | 10,608 | 10,562 | 35.7 | | | | |
| | | 9,508 | | | | | | | |
| | 0.001 | 15,312 | 15,241 | 15,115 | 8.0 | | | | |
| | | 15,170 | | | | | | | |
| Compound 2 | 10 | 99 | 106 | (21) | 100.1 | 0.89 | 2.1 | 0.968 | 2.5 |
| 10 mM | | 112 | | | | | | | |
| | 1 | 12,762 | 13,404 | 13,278 | 19.1 | | | | |
| | | 14,046 | | | | | | | |
| | 0.1 | 17,625 | 21,833 | 21,707 | −32.2 | | | | |
| | | 26,041 | | | | | | | |
| | 0.01 | 21,576 | 20,478 | 20,352 | −23.9 | | | | |
| | | 19,379 | | | | | | | |
| | 0.001 | 17,548 | 16,934 | 16,808 | −2.3 | | | | |
| | | 16,319 | | | | | | | |

### Example 3

### Comparison Compound 2 with FDA-Approved Anti-HIV Drugs

The comparison of compound 2 (FIG. 1B) with existing HIV drugs on the market is shown in Table IV.

TABLE IV
Comparision of Compound 2 with FDA-approved anti-HIV drugs

| | | RT | Protease | | EC50 | Toxicity | Oral |
|---|---|---|---|---|---|---|---|
| Drug | Company | Inhibitor | Inhibitor | Virus Strain | (μM) | (μM) | Bioavailability |
| Compound 2 | C Sixty | yes | yes | HIV-1 | 0.22 | >100 | ˜30% |
| | | | | (wild type) | | | |
| | | | | M184V* | 0.052 | | |
| AZT | Glaxo | yes | | HIV-1 | 0.05 | >50 | — |
| | | | | (wild type) | | | |
| | | | | M184V | 0.01 | | |
| 3TC | Glaxo | yes | | HIV-1 | 0.18 | >363 | — |
| | | | | (wild type) | | | |
| | | | | M184V | >100 | | |
| Saquinavir | Roche | | yes | HIV-1 | 0.001- | >10 | ˜4% |
| | | | | (wild type) | 0.030 | | |
| | | | | M184V | | | |
| Indinavir | Merck | | yes | HIV-1 | ˜0.1 | >400 | |
| | | | | (wild type) | | | |
| | | | | M184V | — | | |
| Ritonavir | Abbot | | yes | HIV-1 | 0.045 | >57 | ˜78% |
| | | | | (wild type) | | | |
| | | | | M184V | 0.8 | | |
| Nelfinavir | Agouron | | yes | HIV-1 | 0.031- | 23 | 52% |
| | | | | (wild type) | 0.043 | | |
| | | | | M184V | 0.4 | | |
| Amprenavir | Vertex | | yes | HIV- | 0.054 | 89 | — |
| | | | | (wild type) | | | |
| | | | | M184V | 0.5 | | |

*common RT mutant (10-20 passages)

REFERENCES

1. H. W. Kroto, J. R. Heath, S. C. O'Brien, R. F. Curl, and R. E. Smalley, Nature (London), 318, 162 (1985).

2. Buckminsterfullerenes, W. E. Bullups and M. A. Ciufolini, Eds, John Wiley & Sons, New York (1993).

3. A. Yamasaki, T. lizuka, E. Osawa, Full. Sci Tech, 3, 529 (1995).

4. D. Heymann, T. E. Yancey, W. S. Wolbach, M. H. Thiemens, E. A. Johnson, D. Roach, S. Moecker, Geochima Cosmochicia Acta, 62, 173 (1998).

5. L. Becker, R. J. Preda, and T. E. Bunch, Proc. Natl. Acad. Sci. USA, 97, 2979 (2000).

6. A. W. Jensen, S. R. Wilson, and D. I. Schuster, Biorg. Med Chem. Lett., 4, 767 (1996).

7. S. R. Wilson, “Biological Aspects of Fullerenes,” in Fullerenes: Chemistry, Physics, and Technology, K. M. Kadish and R. S. Ruoff, Editors, p. 437-466, John Wiley & Sons, New York (2000).

8. I. Lamparth, A. Herzog, and A. Hirsch, Tetrahedron, 52, 5065 (1997).

9. M. Brettreich and A. Hirsch, Tetrahedron Lett, 2731 (1998).

10. L. Y. Chiang, F-J. Lu, J-T. Lin, J. C. S. Chem. Commun, 1283 (1995).

11. C. F. Richardson, D. I. Schuster, and S. R. Wilson, Org Lett., 2, 1011 (2000).

12. H. H. Ku, W. L. Cleveland, and B. F. Erlanger, J. Immunol., 139, 2376 (1987).

13. B.-X. Chen, S. R. Wilson, M. Das, D. J. Coughlin, and B. F. Erlanger, Proc. Natl. Acad. Sci. USA, 95, 10809 (1998).

14. B. C. Braden, F. A. Goldbaum, B.-X. Chen, A. N. Kirschner, S. R. Wilson, and B. F. Erlanger, Proc. Natl. Acad. Sci. USA, 97, 12193-12197 (2000).

15. (a) S. H. Friedman, D. L. DeCamp, R. P. Sijbesma, G. Srdanov, F. Wudl and G. L. Kenyon, J. Am. Chem. Soc. 115, 6505 (1993); (b) R. Sijbesma, G. Srdanov, F. Wudl, J. A. Castoro, C. Wilkins, S. H., D. L. DeCamp and G. L. Kenyon, ibid., 115, 6510 (1993); (c) R. F. Schinazi, R. Sijbesma, G. Srdanov, C. L. Hill and F. Wudl, Antimicrob. Agents and Chemotherapy 37, 1707 (1993).

16. R. F.Schinazi, A. McMillan, A. S. Juodawlkis, J. Pharr, R. Sijbesma, G. Srdanov, J. C. Hummelen, F. D. Boudinot, C. L. Hill, and F. Wudl, Proc. Electrochem. Soc., PV 94-24, 689 (1994).

17. P. Rajagopalan, F. Wudl, R. F. Schinazi and F. D. Boudinot, Antimicrob. Agents and Chemotherapy, 40, 2262 (1996).

18. D. I. Schuster, S. R. Wilson and R. F. Schinaz, Bioorg. Med Chem. Lett. 6, 1253 (1966).

19. Nacsa, J; Segesdi, J; Gyuris, A; Braun, T; Racusch, H; Buvari-Barcza, A; Bereza, L; Minarovitz, J; Molnar, J; Full. Sci. Tech., 5(5) 969 (1997).

20. S. H. Friedman, P. S. Ganapathi, Y. Rubin and G. L. Kenyon, J. Med. Chem. 41, 2424 (1998).

21. M. Brettreich. and A. Hirsch, Tetrahedron Lett. 39, 2731(1998).

22. F. Moussa; N. Gharbi, M. Pressac, V. Tomberli, T. Da Ros, M. Brettreich, M. Hadchouel, B. Arbelle, F. Trivin, R. Céolin, A. Hirsch, M. Prato, H. Swarc and R. V. Bensasson, Proceeding Electrochem. Soc. Meeting, Nazario Martin, Michele Maggini and Dirk M. Guldi, Eds., 240 (2000).

23. F. Moussa, F. Trivin, R. Céolin et al., Fullerene Sci. Tech., 4, 21 (1996).

24. D. Tomalia, “Dendrons, Dendrimers, and Dendrigrafts: Structure Controlled Polymer Architectures and Self-Assembling Systems”, Non-Viral Genetic Therapeutics—Advances, Challenges and Applications for Self-Assembling Systems, p. 1.2.1-1.2.3.

25. Tomalia, D. A., Naylor, A. M., Goddard, W. A., III Ang. Chem. Int. Ed. Engl. 29 138-175 (1990).

What is claimed is:

1. A composition comprising:
(a) a pharmaceutically effective amount of water-soluble fullerene compounds of one or more of the formulae I-VI: 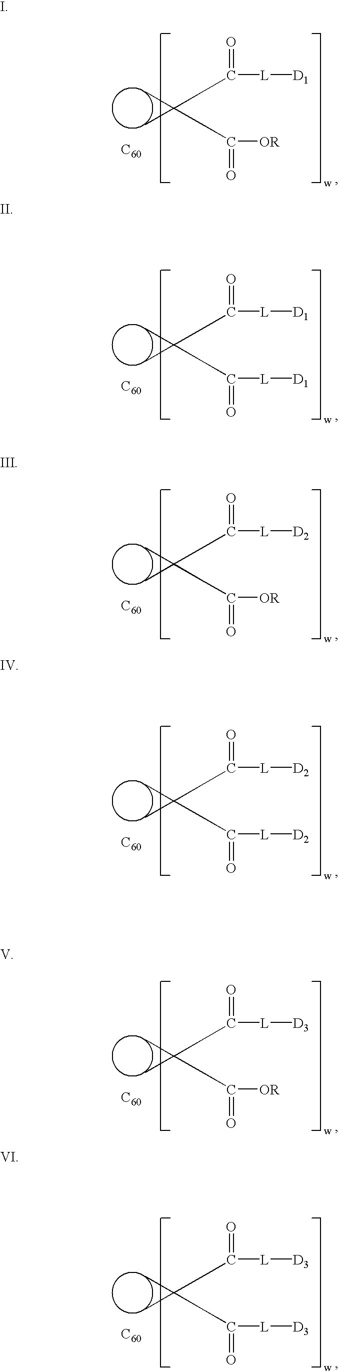
where L is a linker of the formula 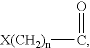
X is NH or O,
R is H or lower alkyl having 1-4 carbon atoms,
n is 1-6, and
D₁ is a dendron of the formula 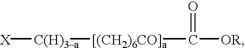
D₂ is a dendron of the formula 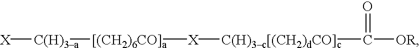
D₃ is a dendron of the formula 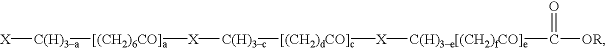
a, c and e are the same or different and each is 1 or 2, b, d and f are the same or different and each is 1-6 and w is 1-6, or mixtures thereof, or water soluble salts thereof, and
(b) a pharmaceutically acceptable carrier.

2. The composition of claim 1, wherein the composition has virucidal properties against a human retrovirus.

3. The composition of claim 2, wherein the retrovirus is a strain of HIV.

4. The composition of claim 1, wherein the structure of the fullerene derivative is:

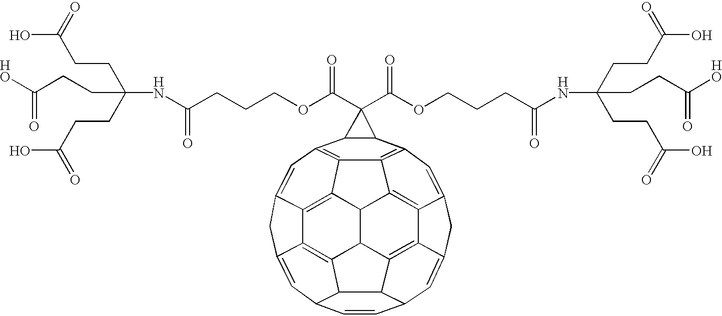

5. The composition of claim 1, wherein the structure of the fullerene derivative is:

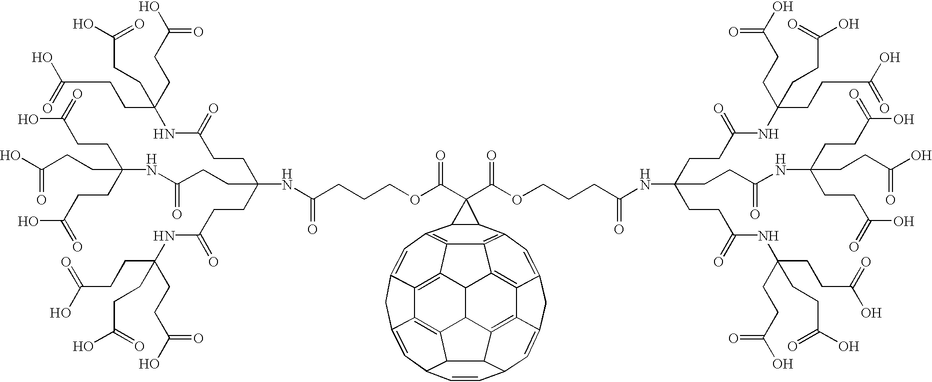

6. The composition of claim 1, wherein the structure of the fullerene derivative is:

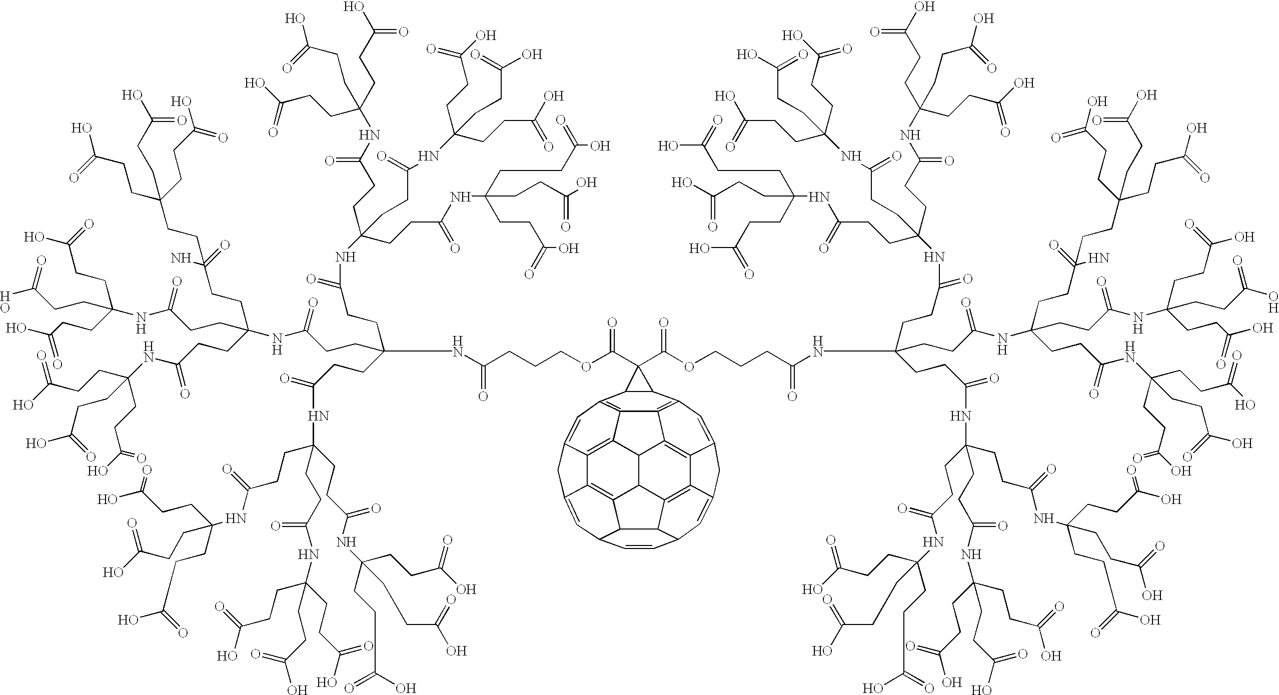

7. A method for the treatment of humans infected with a human retrovirus comprising administering to a patient infected with said retrovirus a composition comprising:
(a) a pharmaceutically effective amount of water-soluble fullerene compounds of one or more of the formulae I-VI: 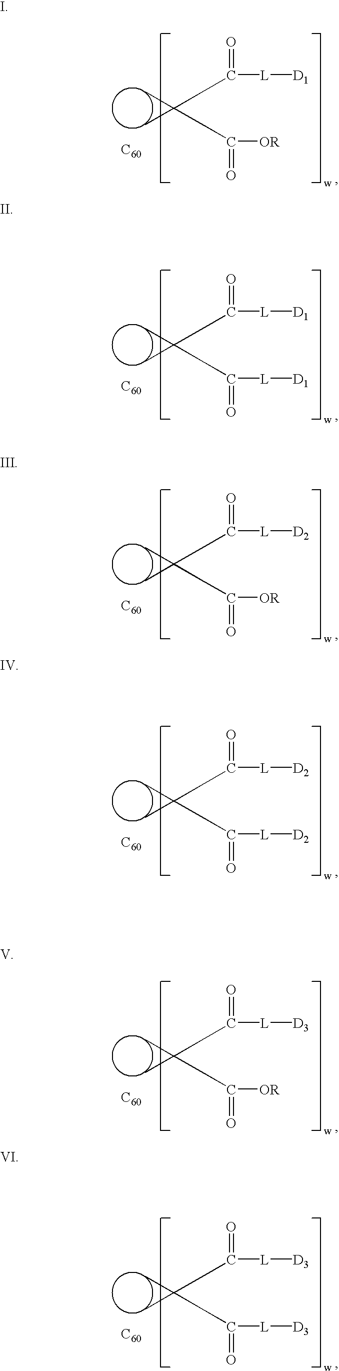
where L is a linker of the formula 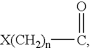
X is NH or O,
R is H or lower alkyl having 1-4 carbon atoms,
n is 1-6, and
D₁ is a dendron of the formula 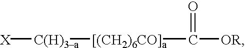
D₂ is a dendron of the formula 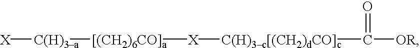
D₃ is a dendron of the formula 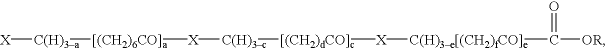
a, c and e are the same or different and each is 1 or 2, b, d and f are the same or different and each is 1-6 and w is 1-6, or mixtures thereof, or water soluble salts thereof, and
(b) a pharmaceutically acceptable carrier.

8. A method as recited in claim 7 wherein said fullerene derivative is:

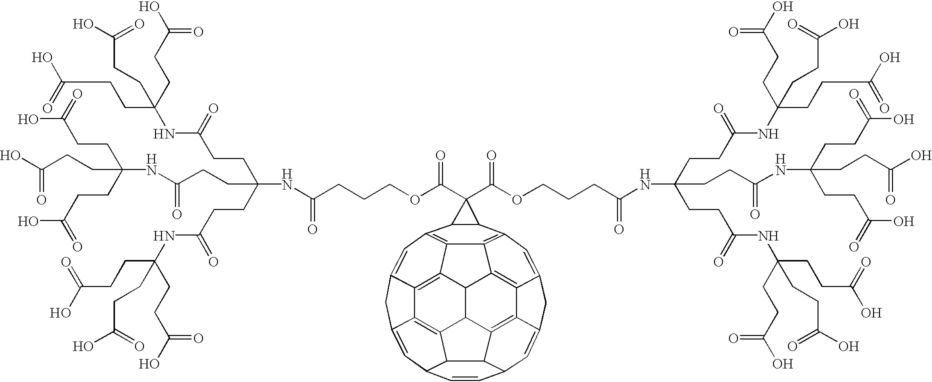

9. A method as recited in claim 7, wherein said retrovirus is HIV.

10. A method as recited in claim 9 wherein the strain of HIV is M184V, HIV-1/LAI, xxBRU, M184V/T215Y or 215/41.

11. A method as recited in claim 9 wherein the strain of HIV is 3TC resistant.

12. A method as recited in claim 9 wherein the strain of HIV is AZT/3TC resistant.

13. A method as recited in claim 9 wherein the strain of HIV is AZT resistant.

14. A method as recited in claim 7 wherein said composition is administered to said patient in a dosage of from 5-25 mg/day.

15. A method as recited in claim 7 wherein said fullerene derivative is [chemical structure]
